(19) United States
(12) Patent Application Publication
(10) Pub. No.: US 2008/0102106 A1
(43) Pub. Date: May 1, 2008

(54) Wound Healing Composition Comprising Substances From Diptera Larvae

(75) Inventors: Jochen D'Haese, Velbert (DE); Heinz Mehlhorn, Neuss (DE); Thomas Ruzicka, Dusseldorf (DE); Jurgen Schmidt, Dusseldorf (DE); Helger Stege, Freiburg (DE)

Correspondence Address: RANKIN, HILL & CLARK LLP, 925 EUCLID AVENUE, SUITE 700, CLEVELAND, OH, 44115-1405, US

(73) Assignee: ALPHA-BIOCARE GMBH, Dusseldorf (DE)

(21) Appl. No.: 11/814,193

(22) PCT Filed: Dec. 1, 2006
(86) PCT No.: PCT/EP06/69210

§ 371 Date: Aug. 28, 2007

(30) Foreign Application Priority Data
Dec. 20, 2005 (DE) 102005061246.6

(51) Int. Cl. A61K 35/64; A61K 9/12; A61L 15/16; A61P 43/00

(52) U.S. Cl. 424/446; 424/43; 424/538

(57) ABSTRACT

Disclosed is a wound healing composition comprising a mixture of substances that are obtained from diptera larvae, are heat-resistant at 100° C., and have molecular weights ranging from 500 to 10,000 Dalton. Also, disclosed are the use of said composition as well as a method for the production thereof.

The present invention relates to a composition for wound healing comprising substances from diptera larvae.

Many factors and processes play a role in wound healing. Both in acute as well as in chronic wounds, the healing process progresses in three successive phases, the exsudative, the proliferative, and finally the reparative phase. In the exsudative phase, which is clinically characterised by a wound edema and traumatic pain, reactions having vasoconstrictive and haemostaseogical effects are in the foreground. In the process, macrophages, neutrophile granulocytes and lymphocytes are attracted. By means of so-called tissue debridement, cell debris and necrotic tissue are removed by phagocytosis. The proliferative phase of wound healing begins with the immigration of fibroblasts and vascular endothelial cells. The newly formed tissue mass grows, and an increased release of cytokines and growth factors takes place which in turn promote the proliferation of cells and the regeneration of vessels. The extracellular matrix is modified in this phase, and finally, a well-capillarised granulation tissue develops containing macrophages, fibroblasts and mast cells. Ceratinocytes immigrate into the wound in the final epithelisation and reparative phase. While the capillary density now decreases, the collagen content rises, consolidating the scar.

However, if the progress of complex processes of wound healing is impaired, the healing can be delayed considerably. If this process extends beyond 6 weeks, this is considered a chronic wound healing disorder. Healing disorders often occur as a consequence of diseases, in particular diabetes mellitus and immunodepressions, as the result of varicosis or in certain bacterial infections. In order to improve the wound healing, measures must be taken for accelerating the processes in the three phases of wound healing mentioned. In particular, the primary cause of the delayed wound healing—the deficient formation of granulation tissue—must be eliminated, which is caused either by reduced debridement of the wound or by excess formation of cell detritus.

### PRIOR ART

Surgical procedures are suitable as measures for cleaning necrotically or fibrinously covered acute or chronic wounds, however, new traumas are caused in the process which can lead to further delay in wound healing, in particular in chronic wounds. In the case of infected wounds, these measures can not be carried out at all, or only with the application of antibiotics. In the vacuum sealing method, the wounds are occlusively covered while exposed to suction, and the covering layers are removed by means of negative pressure. This method is effective but, as a rule, requires immobilization of the patient. Pharmacological methods are based on the use of proteolytic enzymes. In most marketed preparations, the enzymes are obtained from bacteria, monocellular animals or cattle. In practical application, however, the efficacy of the preparations is often low, inter alia, because the half-life of the enzymes is too short, or because the target substances to be broken down in the necrotic tissue do not correspond to the action spectrum of the enzymes. Due to their low efficacy or because of lack of proven efficacy, a number of commercial products are not available on the market anymore (as of December 2005).

The use of living fly maggots of the genus Lucilia sericata is known as a means for wound healing. This mode of therapy, wherein maggots are brought onto the wounds, has been known for centuries from folk medicine—the maggots of the flies feed on necrotic tissue, but not on healthy tissue. The maggots of Lucilia sericata lyse the necrotic tissue by means of substances secreted per os and then imbibe the cell pulp.

With regard to the use of living maggots in wound healing, there are problems in three areas. Thus, many people are revolted by the crawling maggots, which, in addition, can also cause considerable pain when clinging to the wound with their mouthparts. It is difficult to breed living fly maggots that are free from potential pathogens such as bacteria or fungi in order to avoid microbial infections of the patient. The fly maggots must also be kept sterile also during transport from the laboratory to the patient. Another problem arises from the logistics of the maggots, because fly maggots cannot be “fixed” in this stage, but continue to grow and pupate after a few days. Therefore, it is difficult to have larvae of a suitable size ready for use when an appropriate patient arrives in the hospital and to keep them ready for each new application.

From DE 10149153 A1, DE 10138303 A1, DE 19901134 C2 as well as DE 103 27 489 A1 it is known that, instead of living fly maggots, secretions or extracts obtained from the maggots can be used in the therapy of wounds.

DE 103 27 489 A1 discloses homogenates, extracts and constituents thereof from diptera pupae and their use in the treatment of wounds. Compositions from larvae are not disclosed. In addition, (1) neither discloses the molecular weight of the extracted substances, nor their thermal stability. On the contrary, the extracts according to the teaching of this document are obtained and stored under continuous cooling.

JP 06 199898 discloses an extract from housefly pupae comprising peptides having a molecular weight of 3350±900 daltons. Thermal stability is not mentioned. In addition, extracts according to (2) are not used for wound healing, but rather as tyrosinase inhibitor, skin lightening agents, preservatives for foodstuffs or insecticides.

CN-A 123131 (Derwent Abstracts accession number 2000-08779) also does not disclose a composition for wound healing, but rather an extract from fly larvae or pupae for strengthening immunity and tumour inhibition. The molecular weight of the constituents of the extract is not defined in this document.

WO 2006/066619 is a document published after the priority date of the present application. The document discloses extracts from diptera pupae, not from larvae. In addition, neither the molecular weight nor a thermal stability of the extracts are disclosed. On the contrary, the extracts according to the teaching of this document are continuously cooled.

Though WO 03/013557 discloses extracts from diptera larvae for wound healing, neither does it disclose the claimed molecular weight, nor the thermal stability. This document, too, expressly calls for the extracts to be cooled continuously.

During the search for the active substances of the maggot extracts, it was already surmised in the literature that allantoin, a product of the purine metabolism, or urea and/or ammonium could possibly accelerate wound healing (Robinson W: Stimulation of healing in non-healing wounds by allantoin occurring in maggot secretions and of wide biological distribution. J Bone Joint Surg 1953; 17:287-271. Robinson W: The healing properties of allantoin and urea discovered through the use of maggots in human wounds. Ann Rep Smithsonian Institution, Washington, DC US Government Printing Office. 1938, S. 451-460. Robinson W, Baker F C: The enzyme urease and occurrence of ammonia in maggot infected wounds. J Parasitol 1939; 25:149-155). In more recent times, allantoin and urea have been used as cosmetic agents for numerous skin care products. However, because of the many cases of use of these substances in humans, in particular from many dermatological studies concerning possible skin care properties of these substances, it may be considered confirmed that allantoin, urea and ammonium do not have any wound healing effect in contrast to the maggot extracts.

Because of several studies, it is assumed that maggot extracts have a favourable effect on wound healing due to antimicrobial action. An alkaline pH value of the wounds when colonised with maggots due to ammonium or ammonium bicarbonate was considered the reason for an accelerated wound healing (Messer F C, McClellen R H: Surgical maggots. A study of their functions in wound healing. Journal of Laboratory and Clinical Medicine 1935; 20:1219-1226). Wounds can be infected with bacteria, yet also in many poorly healing wounds, colonisation with pathogenic germs is relatively small (Falanga V: Wound healing and its impairment in the diabetic foot. Lancet 2005, 366:1736-1743). Therefore, it must be assumed that the microbicidal effect of the maggot secretions cannot generally explain the accelerated wound healing. Furthermore, in the method according to the invention, the ammonium is removed during lyophilisation, so that this cannot play any role in the wound healing.

Cleaning the wound of dead cells and cell debris, debridement, is an important phase at the start of the healing process. Various proteases are used for dissolving the cell debris and various proteins of the extracellular matrix. WO 01/31033 claims obtaining a particular protease having a molecular mass of approximately 25 KDa from Lucilia sericata maggots and its use in wound healing. Defined proteases are also proposed for use in patients (Chambers L, Woodrow S, Brown A P. Degradation of extracellular matrix components by defined proteinases from the greenbottle larva Lucilia sericata used for the clinical debridement of non-healing wounds. Br. J. Dermatol 2003; 148:14-23). However, there are no controlled clinical studies on the treatment of wounds with proteases from diptera, and at the moment (December 2005), no commercial preparations having proteases from diptera for the treatment of wounds are available.

One serious problem with respect to the use of total extracts from diptera for use in wounds lies in the fact that a number of proteins in these extracts can induce immune reactions in wounds, in particular in the case of repeated, long-term use. As a consequence of these immune reactions, inflammation of the wounds and a generalised allergic reaction of the body may occur. DE 10327489 A1 describes an extract from fly maggots, which, however, contains many immunogenic proteins with a potential for being harmful to health.

An important and hitherto unsolved task consists of obtaining, from the extract of diptera, such substances that, firstly, stimulate wound healing, that, secondly, have as little as possible immunological side effects, that, thirdly, can be produced free from contamination with microorganisms, and that, fourthly, can be formulated as a medicament that can be standardised and dosed well.

In view of this background, the object of the invention is to provide a composition for wound healing that does not exhibit the above-mentioned disadvantages and solves the problems mentioned.

### DISCLOSURE OF THE INVENTION

Surprisingly, it has now been found that, after the complete removal of all proteolytic activity of fly maggot extracts by heat treatment, whereby the proteases are denatured, and also in the case of prior ultrafiltration for the purpose of removing all proteins having a molecular mass greater than 10 KDa, the preparations nevertheless cause an excellent wound healing when used in patients. Thus, proteases are not essentially required for wound healing—as has often been assumed. The invention was completed on the basis of this result.

Surprisingly, it has now been found that the isolates according to the invention, obtained from larvae of diptera, are able to remedy the aforementioned disadvantages of the current therapeutic measures in wound healing.

The above-mentioned object is solved by a composition for wound healing according to claim 1. Preferred embodiments of the composition are defined in the claims 2 to 18. Furthermore, the invention relates to the use of a composition according to the invention for wound therapy, as well as a method for producing a composition according to the invention.

The extracts according to the invention from diptera larvae constitute a significant improvement of the current practiced therapy with living maggots of flies.

It was found that the wound-healing substances can be obtained by means of ultrafiltration through a membrane with an exclusion limit of 10,000 dalton. Surprisingly, it was also shown that the active substances are retained in a second ultrafiltration with a membrane having an exclusion limit of 500 dalton. An accelerated wound healing could only be achieved by only using the fraction containing substances having molecular masses of between 500 and approximately 10,000 dalton. Obviously, neither proteins having a higher molecular mass, inter alia proteases, nor very small organic or inorganic substance are important for wound healing.

Furthermore, it is possible to treat the cleaned-up isolates with heat, in particular, to autoclave them at 121° C., and thus to safely make them sterile.

Furthermore, it is possible to dry the preparations without loss of activity, preferably lyophilise them in the process, to store them, and reconstitute them only when needed. To the treating physician or the patient, such a storable ready-made preparation constitutes a medicament that is always available.

Suitable species for the production of the preparations are, for example, from the genus Sarcophaga S. camaria, from the genus Lucilia: L. sericata, P. regina from the genus Phormia, C. erythrocephala from the genus Calliphora and M. domestica from the genus Musca. These species can be bred easily and in large quantities in the laboratory for obtaining the extracts.

The extracts or isolates obtained are stored in suitable carrier media, for example physiological saline solutions, sterile electrolyte solutions, albumin solutions, oil or fats, prior to processing.

The invention also relates to a medicament, characterised by an active content of the extracts according to the invention or constituents thereof with a wound-healing action, together with a pharmaceutically suitable physiologically compatible carrier substance, additive and/or other active or auxiliary substances. Because of the pharmacological properties, the inventive preparations are suitable for the therapy of superficial or deep chronic and acute wounds of any genesis.

The term “chronic and acute wounds of any genesis” are understood to be, for example, wounds such as surgical wounds that are supposed to heal intentionally or unintentionally per secundam, cut injuries, stab injuries, abrasions, bite injuries or shot injuries, as well as other wounds that cannot be treated per primam by means of a surgical suture or a primary wound closure. In addition, the term acute wounds also signifies all wounds which cannot heal per primam due to a microbial infection, and all wounds whose manifestation is 4 weeks and less. Chronic wounds are all injuries that are accompanied by the break-up of the integrity of the epithelium and are manifest for more than 4 weeks. In particular, poorly healing wounds based on a diabetes mellitus, a varicosis or venous thrombosis, a rheumatic disorder, an occlusive arterial disease, a disease of a lymphatic vessel, haematological diseases and during or after infections of wounds are meant with this term.

The invention also relates to a method for producing a medicament characterised by the inventive extracts or constituents thereof having wound healing activity being brought into a suitable form of administration with a pharmaceutically suitable and physiologically compatible carrier, and, if necessary, further suitable active substances, additives or auxiliary substances.

Application of the medicaments according to the invention is usually done topically. Suitable pharmaceutical compositions for topical use on the skin are at hand, preferably, as an ointment, solution, suspension, cream, powder, liposomal or oleosomal formulations, gel, lotion, paste, spray, aerosol or oil. Vaseline, lanolin, polyethylene glycols, alcohols and combinations of two or more of these substances may be used as carriers. The extracts or enzyme isolates according to the invention are generally present in a concentration of 0.1% by wt to 100% by wt of the composition, preferably of 1.0% by wt to 60% by wt.

Transdermal administration is also possible. Suitable pharmaceutical compositions for transdermal uses can be present as individual plasters that are suitable for long-term close contact with the epidermis of the patient. In a suitable manner, such plasters contain the extracts or isolates according to the invention, in an aqueous solution that, if necessary, is buffered, dissolved and/or dispersed in an adhesive agent, or dispersed in a polymer. A suitable concentration of the active substance is from about 0.1% by wt to 75% by wt, preferably from 1% by wt to 70% by wt. As a special option, the active substance can be released by electrotransport or iontophoresis, as described, for example, in Pharmaceutical Research, 2: 318 (1986).

Moreover, the extracts according to the invention can also be applied to the wound by means of wound dressings made from gauze, alginates, hydrocolloidal materials, foams and/or silicone dressings that were coated, impregnated or treated with these extracts or enzyme isolates, and are thus capable of releasing the active substances into or onto the wound or wound surface.

Suitable solid or galenical forms of preparation are, for example, granulates, powders, dragëes, tablets, (micro) capsules, suppositories, syrups, juices, solutions, suspensions, emulsions, drops or injectable solutions as well as preparations with protracted release of active substances, during the production of which commonly used auxiliary or carrier substances such as disintegrants, binding agents, coating agents, swelling agents, lubricants, flavouring substances, sweetening agents and solubilizers are used Mention is made of magnesium carbonate, titanium oxide, lactose, mannitol and other sugars, talcum, lactoprofein, gelatine, starch, cellulose and its derivatives, animal and vegetable oils such as liver oil, sunflower oil, peanut oil, or sesame oil, polyethylene glycol and solvents such as sterile water and mono- and polyhydroxilic alcohols, such as glycerine as frequently used auxiliary substances.

Moreover, the extracts according to the invention can also be used in galenical preparations to which active substances that are suitable for debridement, for example enzymes, are added.

The use of a powder or of lyophilisates that are dissolved in a physiological solution (e.g. 0.9% aqueous NaCl) are simple examples. Given sufficient stability, the galenical preparation may also be a solution.

Application of the suitable pharmaceutical preparations is carried out following a mechanical wound cleaning. Mechanical wound cleaning is done, for example, by means of a bath or a rinsing of the wound with lactated Ringer's solution. After the application of the extracts according to the invention, the wound is optionally covered with hydrocolloidal wound dressings, or with wound dressings coated with diptera extract or with self-adhesive surgical wrap. The change of the bandages with a renewed administration of the extracts or isolates according to the invention each time is carried out daily.

### EXAMPLES

### Production of Preparations According to the Invention

Larvae of the species Musca domestica and/or Calliphora erythrocephala, Lucilia sericata, Phormia regina, Sarcophaga camaria are harvested from fresh, superficially flamed and uncontaminated horse meat or plant extract. The subsequently collected maggots are externally cleaned in several washing steps in sterilised isoosmotic saline solution. The maggots are then crushed and the contents are collected in a carrier medium on ice. Then, the ingredients are homogenised. This is done in several steps by means of ultrasound or mechanical homogenisation, or by adding solvents. After a homogeneous liquid has been obtained, the extract is filtered through a sterile filter having an exclusion limit of 10,000 daltons, e.g., by means of a Vivacell 250 filtering apparatus having a membrane of polyether sulfone 10,000 MWCO. The filtrate is given into a dialysis tube made of a cellulose membrane having an exclusion limit of 500 daltons, and dialysed for three days at 4° C.—in order to avoid antimicrobial destruction—against an aqueous solution containing 0.9% by wt sodium chloride replaced several times. The solution retained in the dialysis tube is aliquoted in suitable containers or distributed onto suitable wound dressings by spraying. The preparations are then sterilised by heating to 100° C. for 30 minutes, or preferably by autoclaving at 121° C. under pressure. Then, the preparations can be stored with cooling. Preferably, however, the preparations are dried or lyophilised under a clean bench in order to make them storable without cooling.

### Example 1

### Production of a Preparation from One Species of Fly

1,500 fly maggots of the species Lucilia sericata were taken from the production cages and collected in the refrigerator until the number has been reached. Then, they were mechanically homogenised and at first centrifuged in a refrigerated centrifuge for two hours. The supernatant was then centrifuged for twelve hours with a Beckman ultracentrifuge. Then, as described above, the supernatant was ultrafiltered, dialysed, autoclaved and stored in portions of 1 ml, 0.2 millilitres per 10 cm² surface of the wound are used.

### Examples of Use Concerning the Wound Treatment in Patients

### Example of Use 1

A 87-year old patient with a postthrombotic syndrome, chronic venous insufficiency and an ulcus cruris venosum on the lower leg that has been in evidence for three years. Despite modern wound treatment, the ulcer had not healed during this time. At the initiation of therapy, there were large-area ulcerations and necroses present. A preparation was used that had been produced, as in example 1, from maggots of the species Lucilia sericata and stored in liquid form at 4° C. Daily use of 0.2 ml per 10 cm² surface of the wound led to a complete debridement and incipient epithelisation within ten days. After eight weeks of therapy accompanied by additional compression, a complete healing had occurred.

Example of use 2

A 72-year old patient with fourth degree chronic venous insufficiency, the ulcer had existed for 18 months and had chronic fibrinous coverings. In the past, neither hydrocolloid bandages nor PU foams could prevent the development of coverings. Healing did not take place. For producing the preparation, ultrafiltered and dialysed extracts from maggots of the species Musca domestica were applied in a dosage of 30 microlitres per cm² wound dressing, then autoclaved, dried under a sterile workbench and finally packaged sterile. Initially, the treatment was carried out by daily change of the wound dressing, and later every second day. During the therapy, a complete debridement and healing of the ulcer occurred within 14 weeks. No side effects were observed. After eight months, the patient had another ulcer requiring treatment. In this case, therapy was also carried out with a wound dressing coated with dried maggot extract. Healing was complete within 9 weeks without any side effects in the form of inflamed immune reactions to the maggot extract according to the invention becoming evident.

1. A composition for wound healing, comprising a mixture of substances derived from diptera larvae, that are thermally stable at 100° C., and have molecular masses of between 500 and 10,000 daltons.

2. The composition of claim 1, obtainable from larvae of the genera of flies Musca, Calliphora, Phormia, Sarcophaga or Lucilla, or mixtures of larvae of two or more species.

3. The composition of claim 1, characterized in that it comes from a species selected from the group consisting of Musca domestica, Calliphora erythrocephala, Sarcophaga canaria, Phormia regina, Lucilla sericata, Lucilla caesar, and combinations thereof.

4. The composition of claim 1, characterized in that the larvae are homogenized mechanically, by the addition of chemicals and/or by ultrasound.

5. The composition of claim 1, characterized in that the homogenates are liberated from constituents not soluble in water and that immunogenic proteins and glycoproteins having molecular masses of more than 10,000 daltons are removed.

6. The composition of claim 1, characterized in that the separation of insoluble constituents and high-molecular substances is carried out by one selected from the group consisting of centrifugation, ultracentrifugation, phase separation, ultrafiltration, chromatographic separation, and combinations thereof.

7. The composition of claim 1, characterized in that a heat treatment is carried out at 60 to 100° C., or that the composition is autoclaved.

8 The composition of claim 1, characterized in that a sterile filtration with a filter having a diameter of pores of 0.1 μm to 0.4 μm is carried out.

9. The composition of claim 1, characterized in that the extracts are used in a concentration of 0.1% by wt to 100% by wt in a galenical composition.

10. The composition of claim 1, characterized in that the extracts are used in a concentration of 0.4% by wt to 60% by wt in a galenical composition.

11. The composition of claim 1 further comprising preservation agents, microbicidal agents, antioxidizing substances, and combinations thereof.

12. The composition of claim 1, further comprising pharmacologically suitable and physiologically compatible auxiliary substances.

13. The composition of claim 1 further comprising one or more proteases that dissolve necrotic tissue for debriding the wound.

14. The composition of claim 1, characterized in that the extract is dried and lyophilised.

15. The composition of claim 1, forming part of a composition selected from the group consisting of ointment, solution, suspension, cream, powder, liposomal formulation, oleosomal formulation, gel, lotion, paste, spray, aerosol or oil.

16. A plaster for application to the skin comprising the composition of claim 1.

17. A wound dressing comprising one selected from the group consisting of gauze, alginates, colloidal materials, foamed substances, silicone dressings, and combinations thereof that are coated or impregnated with extracts or enzyme isolates of substances derived from diptera larvae.

18. The composition according to claim 1 characterised in that the substances from diptera larvae are used in galenical compositions that contain the active substances in an inactive form, and which are then applied into or onto the wound, become active there, or are activated by adding specific substances.

19. A method of treating superficial, deep, chronic, or acute wounds of any origin comprising applying the composition of claim 1.

20. A method of producing the composition of claim 1 comprising combining the thermally stable substances from diptera larvae having a wound healing action together with a carrier that is pharmaceutically suitable and physiologically compatible.
